(19) United States
(12) Patent Application Publication
(10) Pub. No.: US 2026/0216426 A1
(43) Pub. Date: Jul. 30, 2026

(54) MEDICAL FLUID DELIVERY USING SYRINGE

(71) Applicant: KPR U.S., LLC, Mansfield, MA (US)

(72) Inventors: Samuel ALEXANDER, St Louis, MO (US); John BRINKMAN, St Louis, MO (US); Joseph HUDSON, O'Fallon, MO (US); Zachary SANDERS, Maryland Heights, MO (US); Kevin THOMAS, Mansfield, MA (US)

(21) Appl. No.: 19/461,376

(22) Filed: Jan. 27, 2026

Related U.S. Application Data
(60) Provisional application No. 63/750,132, filed on Jan. 27, 2025.

(51) Int. Cl. A61M 5/168; A61M 5/145

(52) U.S. Cl. CPC A61M 5/16804; A61M 5/1456; A61M 2205/3327; A61M 2205/50; A61M 2205/702

(57) ABSTRACT

Apparatuses and methods for an automated fluid delivery system having a flow control device including a pump and a control unit, a fluid administration set, a fluid sensor, and a plunger displacement sensor. The fluid administration set includes a syringe containing the fluid and tubing. The pump generates flow of the fluid from the syringe through the tubing. The fluid sensor detects the fluid in the tubing downstream of the syringe. The plunger displacement sensor is configured to detect linear displacement of the syringe plunger relative to the syringe barrel as fluid flows from the syringe through the tubing. The control unit determines an estimated inner cross-sectional dimension of the syringe barrel based on the linear displacement of the syringe plunger and detected fluid in the tubing downstream of the syringe, and operate the pump based at least in part on the estimated inner cross-sectional dimension of the syringe barrel.

### CROSS-REFERENCE TO RELATED APPLICATION

This application claims the benefit of U.S. Provisional Patent Application No. 63/750,132, filed Jan. 27, 2025 and hereby incorporates by reference herein the contents of this application.

### FIELD

The present disclosure generally relates to medical fluid delivery using a syringe.

### BACKGROUND

Fluid medicine or nutrition can be administered or delivered to a subject or patient using a syringe. An automated fluid delivery system delivers fluid from the syringe, through flexible tubing, and to the patient at a controlled rate of delivery. The amount and rate of fluid delivered to the patient is typically dependent on size parameters (e.g., inner diameter and/or volume) of the syringe being used and the stroke or displacement of the syringe plunger during feeding. Currently, the displacement of the syringe during feeding can be accurately captured using sensors. Thus, the ability of the automated fluid delivery system to accurately deliver the desired flow rate and/or total volume of dispensed fluid is dependent mainly on the accuracy of data relating to the size of the syringe. This is made difficult because the inner diameters of syringes from different manufacturers may have different inner diameters. For example, a 30 mL syringe from one manufacturer may have an inner diameter that is less than an inner diameter of a 30 mL syringe from another manufacturer.

Conventionally, the inner diameters of different volume syringes from different manufacturers are stored in a library (i.e., memory) of the automated fluid delivery system. In one method, the user inputs the size and manufacturer of the syringe into the system, whereby the system may look up a constant value associated with the selected syringe to be used in determining flow rate and/or total volume of dispensed fluid based on displacement of the syringe plunger. However, this process is possibly prone to both user errors and system errors, leading to inaccurate fluid deliver. For example, the user may incorrectly confirm or enter the wrong syringe size and/or manufacturer. Moreover, the constant value in the library may be out of date if the manufacturer changes its syringe. Moreover still, manufacturing tolerances or errors between different manufacturing lots may cause the constant value to be unacceptably inaccurate.

### SUMMARY

In some aspects, an automated fluid delivery system for delivering medical fluid to a patient includes a flow control device, a fluid administration set, a fluid sensor, and a plunger displacement sensor. The flow control device includes a pump and a control unit in communication with the pump. The fluid administration set includes a syringe containing the medical fluid, and tubing in fluid communication with the syringe. The syringe includes a barrel and a plunger. The fluid administration set is operatively coupled to the pump so that the pump generates flow of the medical fluid from the syringe through the tubing. The fluid sensor is in communication with the control unit. The fluid sensor is configured to detect the medical fluid in the tubing at a location downstream of the syringe. The plunger displacement sensor is in communication with the control unit. The plunger displacement sensor is configured to detect linear displacement of the plunger relative to the barrel as the pump generates flow of the medical fluid from the syringe through the tubing. The control unit includes a processor and memory storing processor-executable instructions that, when executed by the processor, cause the processor to: determine an estimated inner cross-sectional dimension of the barrel of the syringe based on the linear displacement of the plunger and the fluid sensor detecting the medical fluid in the tubing at the location downstream of the syringe, and operate the pump based at least in part on the estimated inner cross-sectional dimension of the barrel of the syringe.

In some aspects, a flow control device for an automated fluid delivery system configured to deliver medical fluid to a patient includes a pump, a fluid sensor, a plunger displacement sensor, and a control unit. The pump is configured to be operatively coupled to a fluid administration set including a syringe containing the medical fluid and tubing in fluid communication with the syringe. The fluid sensor is in communication with the control unit. The fluid sensor is configured to detect the medical fluid in the tubing at a location downstream of the syringe. The plunger displacement sensor is in communication with the control unit. The plunger displacement sensor is configured to detect linear displacement of the plunger relative to the barrel as the pump generates flow of the medical fluid from the syringe through the tubing. The control unit is in communication with the pump, the fluid sensor, and the plunger displacement sensor. The control unit includes a processor and memory storing processor-executable instructions that, when executed by the processor, cause the processor to: determine an estimated inner cross-sectional dimension of the barrel of the syringe based on the linear displacement of the plunger when the fluid sensor detects the medical fluid in the tubing at the location downstream of the syringe, and operate the pump based at least in part on the estimated inner cross-sectional dimension of the barrel of the syringe.

In some aspects, a method of estimating inner cross-sectional dimension of a barrel of a syringe for a fluid delivery system, includes pumping, via a pump, a known volume of medical fluid from a syringe through tubing connected to the syringe; detecting, via a plunger displacement sensor, linear displacement of a plunger of the syringe relative to a barrel of the syringe that corresponds to the known volume pumped from the syringe via the pump; determining an estimated inner cross-sectional dimension of the barrel of the syringe based on the linear displacement of the plunger and the known volume of medical fluid pumped from the syringe; and operate the pump based at least in part on the estimated inner cross-sectional dimension of the barrel of the syringe.

In some aspects, a method of estimating inner cross-sectional dimension of a barrel of a syringe loaded in a fluid delivery system includes estimating, using a control unit, a first estimated cross-sectional dimension of the barrel of the syringe loaded in the fluid delivery system. Estimating a first estimated cross-sectional dimension of the barrel is based on a detected first parameter analyzed by the control unit. The method includes estimating, using the control unit, a second estimated cross-sectional dimension of the barrel of the syringe loaded in the fluid delivery system. Estimating a second estimated cross-sectional dimension of the barrel is based on a detected second parameter, different from the first parameter, analyzed by the control unit. The method includes comparing the first and second estimated cross-sectional dimensions to one another. The method includes performing an operation of the fluid delivery system, using the control unit, based on the comparison of the first and second estimated cross-sectional dimensions.

In some aspects, a method of estimating inner cross-sectional dimension of a barrel of a syringe loaded in a fluid delivery system includes estimating, using a control unit, a first estimated cross-sectional dimension of the barrel of the syringe loaded in the fluid delivery system. Estimating a first estimated cross-sectional dimension of the barrel is based on a detected first parameter analyzed by the control unit. The method includes estimating, using the control unit, a second estimated cross-sectional dimension of the barrel of the syringe loaded in the fluid delivery system. Estimating a second estimated cross-sectional dimension of the barrel is based on a detected second parameter, different from the first parameter, analyzed by the control unit. In some aspects, the method includes comparing the first and second estimated cross-sectional dimensions to one another. In some aspects, the method includes performing an operation of the fluid delivery system, using the control unit, based on the comparison of the first and second estimated cross-sectional dimensions.

Additional advantages and novel features of these aspects will be set forth in part in the description that follows, and in part will become more apparent to those skilled in the art upon examination of the following upon learning by practice of the disclosure.

### BRIEF DESCRIPTION OF THE DRAWINGS

FIG. 1 is a perspective view of a fluid delivery system including a flow control device and a fluid administration set;

FIG. 2 is a block diagram of electrical components of the fluid delivery system in communication with a control unit.

FIG. 3 is similar to FIG. 1, but with the syringe, and portions of a cassette of the fluid administrative set removed;

FIG. 4 is an enlarged, partial front elevation view of the flow control device showing a rotor and fluid sensors;

FIG. 5 is a block diagram illustrating the fluid sensors in communication with the control unit;

FIG. 6 is a front perspective view of a syringe holder;

FIG. 7 is a flow diagram of a syringe size detection operation;

FIG. 8 is a schematic of the fluid delivery system to illustrate a volume-based syringe size estimation operation;

FIG. 9 is a flow diagram of a first volume-based syringe size estimation operation;

FIG. 10 is a flow diagram of a second volume-based syringe size estimation operation;

FIG. 11 is a flow diagram of a syringe size confirmation operation; and

FIG. 12 is a flow diagram of a syringe size calibration operation.

Corresponding reference characters indicate corresponding parts throughout the drawings.

### DETAILED DESCRIPTION

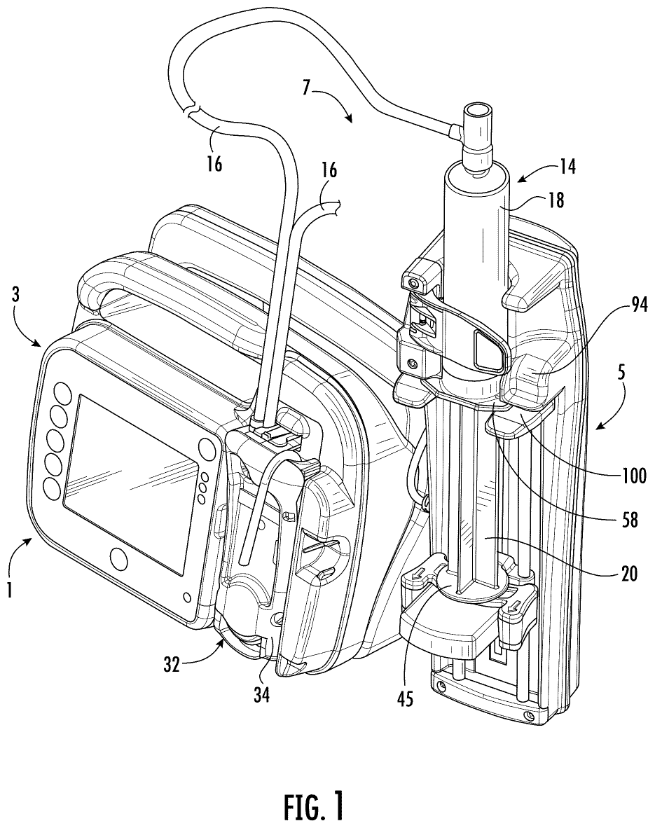

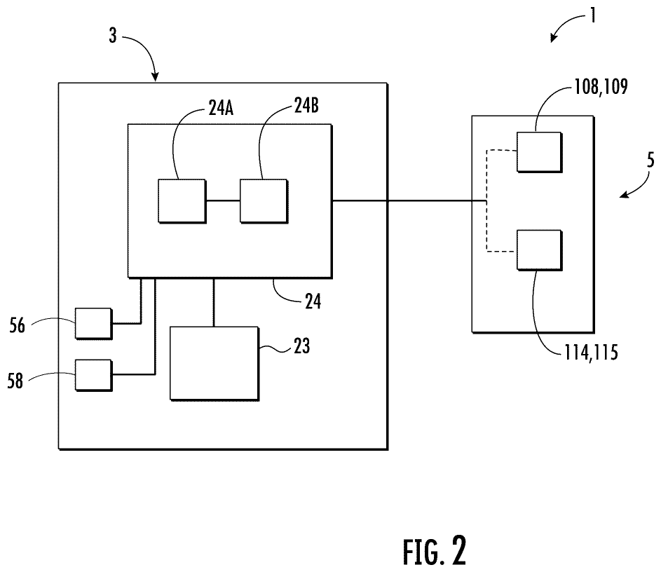

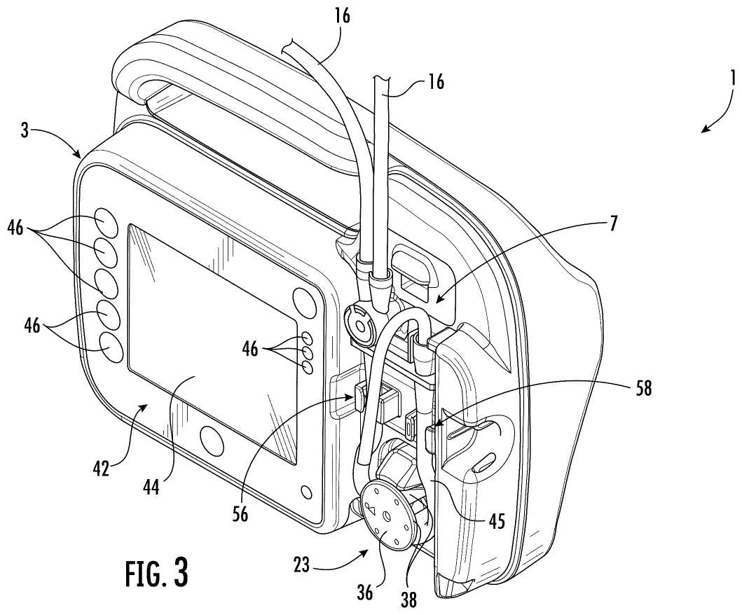

Referring to FIG. 1, one or more aspects of the present disclosure pertain to an automated fluid delivery system, generally indicated at 1. Similar fluid delivery systems are described in U.S. Pat. No. 12,048,830, the disclosure of which is hereby incorporated by reference herein in its entirety. In general, the fluid delivery system includes a flow control device, generally indicated at 3, a syringe holder, generally indicated at 5, and a fluid administration set, generally indicated at 7. The fluid administration set 7 includes a syringe containing medical fluid, generally indicated at 14, and tubing 16 fluidly connected to the syringe 14 for delivering the medical fluid to a subject/patient. Referring to FIGS. 2 and 3, the flow control device 3 includes a pump, generally indicated at 23, operatively couplable to the fluid administration set 7 to impart flow of the medical fluid from the syringe 14 through the tubing 16 to the patient. As used herein, the pump 23 is any device that imparts flow of fluid out of the syringe 14 and through the tubing 16, whereby a plunger 20 of the syringe is moved linearly with respect to a barrel 18 of the syringe. As shown in FIG. 2, the flow control device 3 further includes a control unit 24 including a processor 24A and a memory 24B including processor-executable instructions for execution by the processor.

In one or more aspects, the flow control device 3 includes at least one of a volume-based syringe size estimation operation, a syringe size confirmation operation, and a syringe size calibration operation, each of which is executable by the processor 24A according to stored instructions in the memory 24B. In general, the volume-based syringe size estimation operation estimates an inner cross-sectional dimension of the syringe barrel 18 loaded on the holder 5 based on a displacement of the plunger to dispense a known fluid volume from the syringe. The syringe size confirmation operation alerts the user or inhibits administration of the medical fluid if the control unit 24 determines that an estimated syringe size (e.g., estimated inner cross-sectional dimension) is inaccurate. The syringe size calibration operation calibrates or adjusts operation of the flow control device 3 to more accurately deliver a desired volume and/or flow rate of the medical fluid based on one or more estimated syringe sizes (e.g., estimated inner cross-sectional dimension). For each of these features, the control unit 24 of the flow control device 3 uses sensed operation parameters during operation of the automated fluid delivery system 1 to make decisions and/or calculations affecting operation of the system according to processor-readable instructions (e.g., instruction modules) stored in the memory 24B.

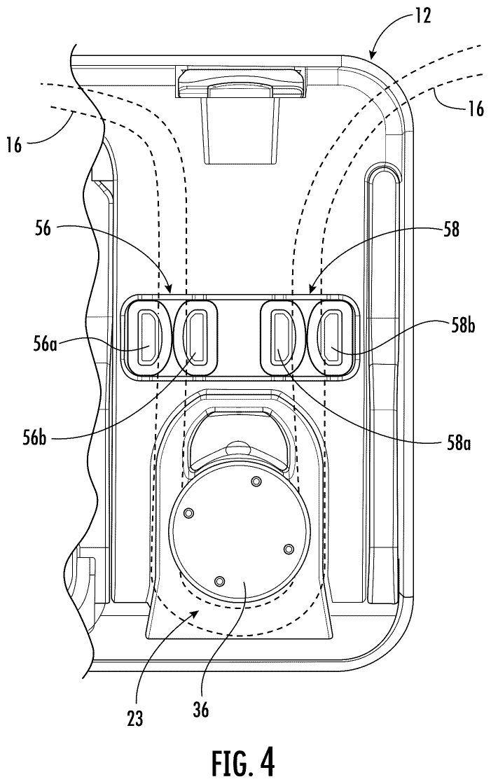

Referring to FIGS. 1 and 3, in the illustrated embodiment, the pump 23 is a peristaltic pump that acts on the tubing 16 and draws the fluid out of the syringe 14. A cassette 32 (FIG. 1) of the fluid administration set 7 is releasably attachable to the flow control device 3. In particular, the cassette 32 is removably received in a cassette recess of the flow control device 3. As shown in FIGS. 3 and 4, when received in the cassette recess, a U-shaped portion of the tubing 16 received in a cassette housing 34 engages a rotor 36 of the peristaltic pump 23. In use, rollers 38 (FIG. 3) of the rotor 36 engage the tubing 16 such that as the rotor is rotated about a rotor axis by a pump motor (not shown), the rollers compress the tubing and push “aliquots” of fluid through the tubing. In general, this type of peristaltic pump 23 for enteral feeding is known in the field and therefore will not be described in detail herein. The pumping action draws fluid from the syringe 14 through the tubing 16 without applying a pushing force to the end of the plunger 20. In another example, not shown, the pump may be a syringe pump, such as a conventional syringe pump, that includes an actuator that pushes on the plunger to actively move the plunger within the barrel to push the medical fluid into the tubing. Other types of pumps may be used in one or more embodiments.

Referring to FIG. 3, the flow control device 3 includes a user interface 42 in communication with the control unit 24. The user interface 42 includes a display 44 and user inputs 46 (e.g., buttons) to enable communication between the control unit 24 and the user. For example, the user interface 42 may enable a user to select a desired flow rate or total dispensed volume for the medical fluid. The display 44 may communicate these selections to the user. The user interface 42 may also enable the control unit 24 to generate alerts and/or alarms to the user.

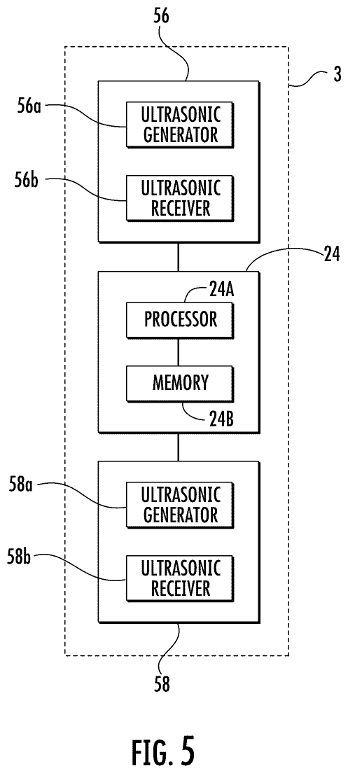

Referring to FIGS. 2-5, at least one fluid sensor is coupled to the flow control device 3: a first fluid sensor, generally indicated at 56, located upstream of the rotor 36; and a second fluid detector, generally indicated at 58, located downstream of the rotor. It is understood that the pump 10 may include a single fluid sensor or more than two fluid sensors without departing from the scope of the present disclosure. In one example, the first and second fluid sensors 56 may be ultrasonic sensors for use in detecting a condition of the fluid in the feeding set 14, although other types of sensors are within the scope of the present disclosure, including, but not limited to, infrared sensors.

Referring to FIG. 4, in the illustrated embodiment, each of the first and second fluid sensors 56, 58, respectively, may include an ultrasonic generator 56a, 58a, respectively (broadly, a sensor signal generator), and an ultrasonic receiver 56b, 58b, respectively (broadly, a sensor signal receiver). For each sensor 56, 58, the ultrasonic generator 56a, 58a and the ultrasonic receiver 56b, 58b are on opposite sides of the tubing 16 in the cassette 32 so that the tubing is received between each pairs of generator and receiver. Each ultrasonic generator 56a, 58a (e.g., an ultrasonic transducer) is configured to receive a drive signal from the control unit 24 (more specifically, the processor 24A), and in response to the drive signal, generate an ultrasonic signal that is transmitted through the tubing 16 toward the corresponding ultrasonic receiver 56b, 58b. Each ultrasonic receiver 56b, 58b (e.g., an ultrasonic transducer) is configured to receive the ultrasonic signal and, in response to the received signal, generate an output signal. The control unit 24 (more specifically, the processor 24A) is configured to receive the output signal and determine and analyze a parameter value of the output signal to determine if fluid is present in the tubing 16. Other types of fluid detection sensors, such as optical sensors, infrared sensors, or other sensors may be used.

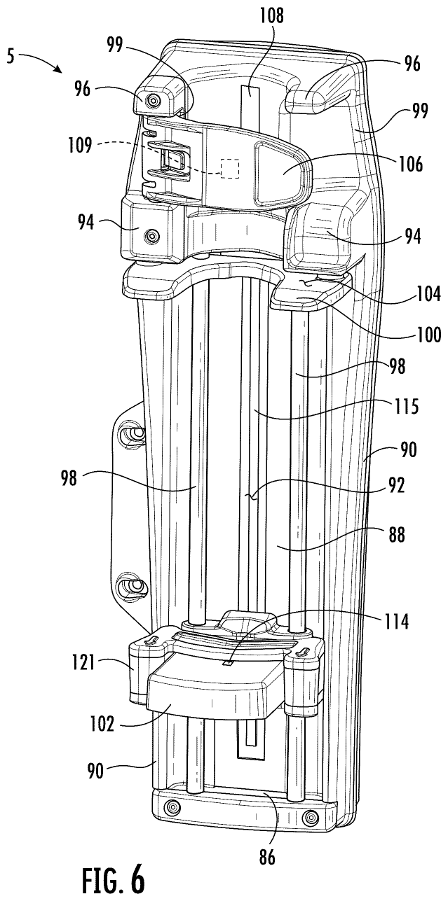

Referring to FIG. 6, the syringe holder 5 includes a floor 86, a rear wall 88 extending from the floor, and opposing side walls 90 extending laterally from the rear wall and away from the floor. The floor 86, rear wall 88, and side walls 90 together define a receiving space 92 for at least a portion of the syringe 14. A first pair of flanges 94 extend from respective side walls 90 of the holder 5 near a top of the holder. Each side wall 90 has a recessed portion 99 above the flange 94 forming a second pair of flanges 96 longitudinally spaced upward from the first pair of flanges 94. A portion of the barrel 18 of the syringe 14 is received between the first pair of flanges 94 and between the second pair of flanges 96. The flanges 94, 96 prevent movement of the barrel 18 in the holder 5 along an axis parallel to the rear wall 88.

A pair of rails or guides 98 may extend between the floor 86 and the first pair of flanges 94. A U-shaped flange plate 100 (broadly, a flange plate) is fixedly disposed at a top end of the rails 98, and a slide plate 102 (broadly, a plunger follower) is disposed around a bottom end of the rails and configured to move or slide along the rails in response to movement of the syringe plunger 20. A gap 104 is formed between the first pairs of flanges 94 and the flange plate 100. The gap 104 is configured to receive a flange 58 of the barrel 18 of the syringe 14 (FIG. 1). The length of the gap 104 is slightly smaller than a thickness of the flange 58. Thus, the flange 58 is held fixed between the flanges 94 and the plate 100 thereby fixing the barrel 18 against longitudinal movement within the holder 5. Further, when the syringe 14 is received in the holder 5, a flange 44 of the plunger 20 may be held between a slide 121 (broadly, a catch) and the slide plate 102. For example, the slide 121 is actuatable to away from the rear wall 88 to provide clearance for the plunger flange 44. The slide 121 can then be moved back toward the rear wall 88 to secure the plunger flange 44 to the slide plate 102. As will be explained in greater detail below, fluid being drawn from the barrel 18 of the syringe 14 causes the plunger 20 to move away from the floor 86. Because the flange 44 of the plunger 20 is captured between the slide 121 and slide plate 102, the movement of the plunger is followed by the slide plate 102 which moves along the rails 98 in response to movement of the plunger.

A door or gate 106 (broadly, a syringe clip) may be pivotably attached between one of the first pair of flanges 94 and one of the second pair of flanges 96 and moveable between an open position to allow the syringe 14 to be received in the receiving space 92, and a closed position for retaining the syringe (i.e., barrel 18) in the receiving space. A sensor 108 may be provided on the holder 5 to detect the position of the door 106 as it is moved between the open and closed positions. For example, a magnet 109 may be located in the gate 106 such that a change in magnetic field is detected based on the angular position of the magnet relative to the sensor 108. Thus, the angular position of the gate 106 is detected as the gate is opened to provide a passage for the syringe barrel 18 to be received in the holder 5, and then closed around the barrel to secure the syringe to the holder. A determination of the outer diameter of the syringe 14 can be made using the angular position of the gate 106 when the barrel 18 is secured in the holder by the gate. In one embodiment, the sensor 108 comprises an angular sensor. Broadly, the sensor 108 and magnet 109 constitute a syringe size sensor.

The holder 5 detects movement of the plunger 20 during pumping of the fluid from the syringe 14. A contact 114 of the potentiometer 115 may be disposed on a movable portion of the holder 5, such as the slide plate 102, so that movement of the slide plate causes the contact to move along the potentiometer 115. Because the barrel 18 is held fixed in the holder 5, as fluid is withdrawn from the barrel, the plunger 20 will move into the barrel. The flange 44 of the plunger 20 is fixed to the slide plate 102 as the plunger moves into the barrel 18 causing the slide plate to move along the rails 98. Therefore, in this embodiment, movement of the contact 114 represents the movement of the plunger 20 relative to the barrel 18 and holder 5 caused by the feeding fluid being drawn out of the syringe 14. Stated another way, the movement of the contact 114 corresponds to the distance which the plunger 20 has advanced into the barrel 18. Broadly, the contact 114 and the potentiometer 115 constitute a plunger displacement sensor.

### OD-Based Syringe Size Estimation Operation

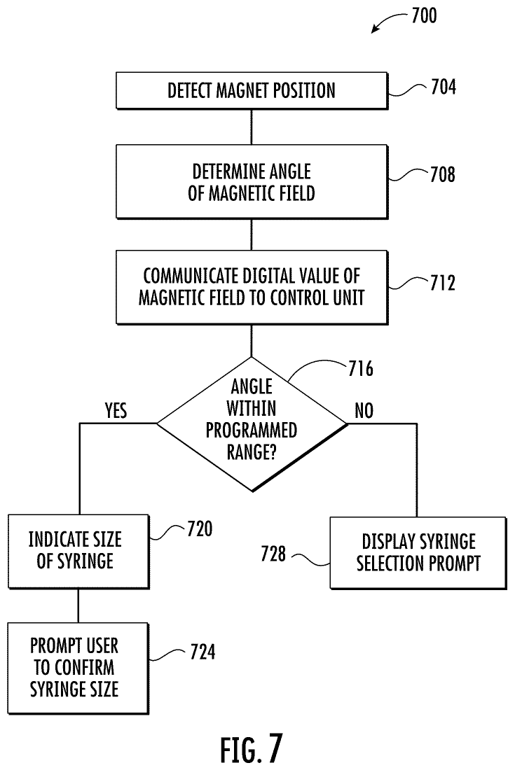

Referring to FIG. 7, an example method 700 of estimating a syringe size using the measured outer diameter of the syringe is disclosed. The method 700 is enacted by the control unit 24 using data received from the sensor 108.

At block 704, the method 700 includes determining a position of the magnet. For example, once the gate 106 has fully secured the barrel 18 of the syringe 14 to the holder 4, a position of a line of demarcation between the north and south pole on the magnet 109 is detected by the sensor 108.

At block 708, the method 700 includes determining an angle of the magnet. For example, an angle of the magnet 109 relative to the syringe holder 5 is then determined by the sensor 108 using integrated Hall devices. For example, the angle of the magnet 109 relative to the rear wall 88 and/or the side walls 90 of the syringe holder 5 can be determined.

At block 712, the method 700 includes communicating a digital value of the magnetic field to the control unit. For example, a proportional digital value of the magnetic field is then communicated to the control unit 24.

At block 716, the method 700 includes determining whether the angle of the magnet falls within a plurality of size ranges. The plurality of size ranges correspond to particular sizes of syringe, and in particular, correspond to particular inner diameters, outer diameters, lengths and volumes of the syringe, the barrel thereof and the plunger designed to be used therewith. For example, the magnetic field signal is compared to statistically generated corresponding digital size range data previously programmed and saved in a memory 24B of the control unit 24. Moreover, the location, position and orientation of the magnet may be selected to correspond to sensing the syringe after it has been properly mounted to the holder 5.

At block 720, the method 700 includes indicating a size of the syringe in response to determining that the angle of the magnet falls within the plurality of size ranges. For example, If the magnetic field angle falls within one of the programmed size ranges, then the control unit 24 determines the size (i.e., volume) of the syringe, such as by using a lookup table. The display screen 44 of the flow control device 3 may display the syringe size that has been detected (e.g., 60 mL syringe).

At block 724, the method 700 includes prompting the user to confirm that the detected syringe size is correct. The control unit 24 may then prompt the user to confirm that the detected syringe size is correct. For instance, the control unit 24 may display a question on the display screen with the option to select “Yes” or “No” to confirm that the detect syringe size is correct.

At block 728, in response to determining that the angle of the magnet does not fall within the plurality of size ranges, the method 700 includes prompting a user to select the syringe size from a list of options or manually input the syringe size. For example, if the magnetic field angle falls outside of the programmed size ranges, the control unit 24 may display a prompt on the display screen for the user to one of select the syringe size from a list of options or manually input the syringe size. For example, the display screen may show four syringe sizes and an “other” option for the user to select from.

The OD-based syringe size estimation operation uses the acquired data from the syringe size sensor 110 (e.g., the sensed outer diameter of the syringe barrel 18) to also determine a correlated inner cross-sectional dimension (e.g., diameter) of the loaded syringe barrel 18. In particular, in one example the memory 24B may include a lookup table that relates data indicating outer diameters of syringe barrels to internal diameters of corresponding syringe barrels 18. Alternatively, a constant may be applied to the outer diameter measurement to calculate a predicted inner cross-sectional dimension of the syringe. In each of these instances, the control unit 24 selects and uses a model syringe from a plurality of model syringes stored in the memory 24B. Each model syringe includes a data set with parameter(s) (e.g., cross-sectional dimensions) based on the detected outer diameter of the loaded syringe 14. The term “model syringe” is used broadly herein: any relationship or calculation using the measured outer diameter of the loaded syringe barrel 18 to determine a predicted inner cross-sectional dimension of the loaded syringe barrel 18 is considered a selection of a model syringe by the control unit 24. Because the inner cross-sectional dimension (e.g., inner diameter) of the barrel 18 is determined/calculated from the detection of the outer diameter of the barrel, the potentiometer 115 can be calibrated so that the movement of the contact 114 indicates the volume of fluid expelled from the syringe 14. In particular, by knowing the inner cross-sectional dimension of the barrel 18 of the syringe 14, in combination with the distance the slide plate 102/plunger 20 has moved, the volume of fluid delivered or displaced from the syringe 14 can be determined by the control unit 24.

### Volume-Based Syringe Size Estimation Operation

In one embodiment, the control unit 24 is configured to calculate an inner cross-sectional dimension of the syringe barrel 18 using a detected dispensed volume of the loaded syringe 14 and a detected linear displacement of the plunger 20. In particular, the memory 24B includes stored processor-readable instructions accessible by the processor 24A that when executed by the processor 24A cause the control unit to perform a volume-based syringe size estimation operation to determine an inner cross-sectional dimension of the syringe barrel.

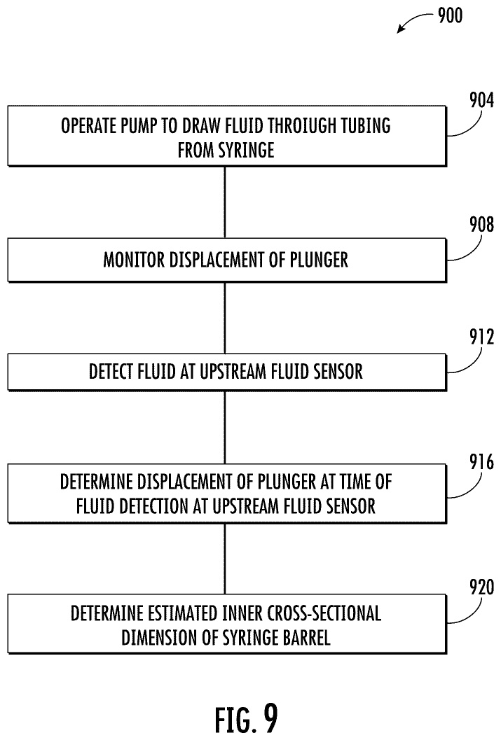

FIG. 9 illustrates an example method 900 for conducting a volume-based syringe size estimation operation. The method 900 is enacted by the control unit 24 using data received from the upstream fluid sensor 56, the downstream fluid sensor 58, and the potentiometer 115.

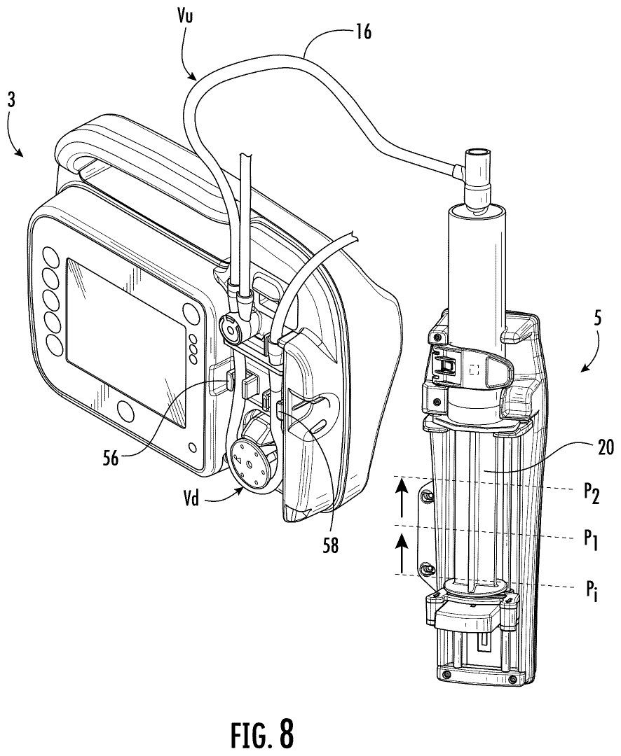

As set forth above, the flow control device 3 includes one or more fluid sensors 56, 58 in communication with the control unit 24 for detecting the presence of fluid in the tubing 16. Referring to FIGS. 8 and 9, at block 904, the method 900 includes drawing fluid from the syringe into an upstream portion of the tubing. For example, the control unit 24 operates the pump 23 to draw fluid from the syringe 14 into an upstream portion of the tubing 16 between an upstream end of the tubing connected to the outlet of the syringe 14 and the upstream flow sensor 56. This is the initial drawing of fluid from the syringe 14, whereby air in the tubing is replaced with the fluid from the syringe. This may also be referred to as initial priming of the upstream portion tubing 16. The internal volume V_{U} (FIG. 8) of the upstream portion of the tubing 16 is known, saved in the memory 24B, and accessible by the processor 24A.

At block 908, the method 900 includes monitoring a position of the plunger. For example, as fluid is being drawn into the tubing, the plunger 20 of the syringe 14 is moved in the barrel 18. The position of the plunger 20 in the barrel 18 is detected and monitored using the plunger displacement sensor 114, 115.

At block 912, the method 900 includes detecting fluid in an upstream portion of the tubing. For example, when the drawn fluid in the upstream portion of the tubing 16 is detected by the upstream fluid sensor 56, the processor 24A receives a fluid-detection signal indicating the presence of fluid. Receiving the fluid-detection signal triggers the processor 24A to retrieve, determine, and/or save the longitudinal position of the plunger 20 in the barrel 18.

At block 916, the method 900 includes determining a displacement of the plunger. For example, referring to FIG. 8, the processor 24A may determine the displacement of the plunger 20 by calculating the difference between an initial longitudinal position of the plunger 20 Pᵢ and the longitudinal position of the plunger P₁ when the upstream fluid sensor 56 detects fluid in the tubing 16. Accordingly, the control unit 24 acquires volume and plunger displacement data that relates the linear displacement of the plunger 20 with the volume Vᵤ pumped out of the syringe barrel 18 to the first flow sensor 56.

At block 920, the method includes estimating an inner cross-sectional dimension of the syringe barrel. For example, using this volume and plunger displacement data determined at block 916, the processor 24A determines a volume-based, first estimated inner cross-sectional dimension of the loaded syringe according to the equation:

$$r = \sqrt{\frac{V}{h π}} ,$$

where r is the calculated inner cross-sectional dimension or radius; V is the volume of the upstream tubing portion; and h is the linear displacement of the plunger.

Alternatively or in addition, the equation can be further honed by solving for r, the interior radius of the syringe barrel, for the volumetric equation of a truncated cone (V=⅓×πh(R²+Rr+r²) to account for the taper of the interior syringe barrel due to an injection molding process.

In one embodiment, the control unit 24 calculates a second estimated inner cross-sectional dimension of the syringe barrel 18 using fluid detection between the two fluid sensors 56, 58. This volume-based operation may be used solely or in addition to the volume-based operation described above.

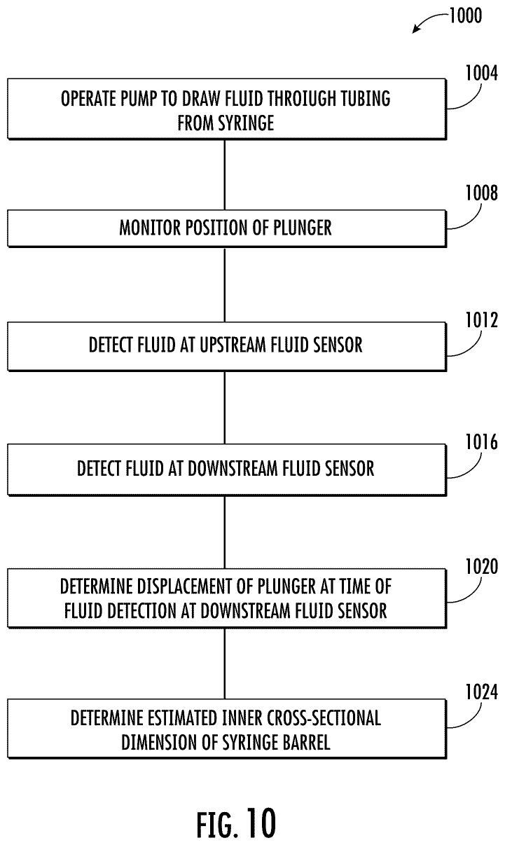

FIG. 10 illustrates an example method 1000 for conducting a second volume-based syringe size estimation operation. The method 1000 is enacted by the control unit 24 using data received from the upstream fluid sensor 56, the downstream fluid sensor 58, and the potentiometer 115.

At block 1004, the method 1000 includes operating the pump to draw fluid from the syringe through the tubing. For example, the control unit 24 operates the pump 23 to draw fluid from the tubing 16 at the first fluid sensor 56 toward the second fluid sensor 58.

At block 1008, the method 1000 includes drawing fluid from the syringe into an upstream portion of the tubing. For example, the control unit 24 operates the pump 23 to draw fluid from the syringe 14 into an upstream portion of the tubing 16 between an upstream end of the tubing connected to the outlet of the syringe 14 and the upstream flow sensor 56.

At block 1012, the method 1000 includes detecting fluid at the upstream fluid sensor. When the drawn fluid in the upstream portion of the tubing 16 is detected by the upstream fluid sensor 56, the processor 24A receives a fluid-detection signal indicating the presence of fluid, similar to what is described at block 912 of the method 900. In response to detecting fluid by the upstream fluid sensor 56, the processor 24A retrieves and/or saves the longitudinal position of the plunger 20 P₁ in the barrel 18 based on the data from the plunger displacement sensor 116. The pump 23 continues to draw fluid through a downstream portion of the tubing 16 (e.g., tubing in the cassette 32). The internal volume V_{D} of the downstream portion of the tubing 16 is known, saved in the memory 24B, and accessible by the processor 24A.

At block 1016, the method 1000 includes detecting fluid at the downstream fluid sensor. For example, when the drawn fluid in the downstream portion of the tubing 16 is detected by the downstream fluid sensor 58, the processor 24A receives a fluid-detection signal indicating the presence of fluid at the second sensor. In response to detecting fluid by the upstream fluid sensor 56, the processor 24A retrieves and/or saves the longitudinal position of the plunger 20 P₂ in the barrel 18.

At block 1020, the method 1000 includes determining displacement of the plunger. For example, the processor 24A may determine the displacement of the plunger 20 by calculating the difference between the first longitudinal position of the plunger 20 P₁ when the upstream fluid sensor 56 detects fluid in the tubing, and the second longitudinal position of the plunger P₂ when the downstream fluid sensor 58 detects fluid in the tubing. Accordingly, the control unit 24 acquires volume and plunger displacement data that relates the linear displacement of the plunger 20 with the volume V_{D} pumped between the first and second fluid sensors 56, 58.

At block 1024, the method includes determining an estimated inner cross-sectional dimension of the syringe barrel. For example, using the volume and plunger displacement data determined at block 1020, the processor 24A calculates a volume-based, estimated inner cross-sectional dimension of the loaded syringe according to the equation:

$$r = \sqrt{\frac{V}{h π}} ,$$

where r is the calculated inner cross-sectional dimension or radius; V is the known volume pumped between the upstream and downstream flow sensors; and h is the linear displacement of the plunger.

Alternatively or in addition, the equation can be further honed by solving for r, the interior radius of the syringe barrel, for the volumetric equation of a truncated cone (V=⅓×πh(R²+Rr+r²) to account for the taper of the interior syringe barrel due to an injection molding process.

### Syringe Size Confirmation Operation

In one embodiment, the control unit 24 performs an automated syringe size confirmation operation to confirm that estimated syringe sizes can be used by the control unit during operation to suitably and accurately deliver a selected volume and or flow rate of fluid to the patient. In particular, the memory 24B includes stored processor-readable instructions accessible by the processor 24A that when executed by the processor 24A cause the control unit to perform a syringe size confirmation operation to determine which (if any) of the estimated syringe sizes can be used by the control unit during operation.

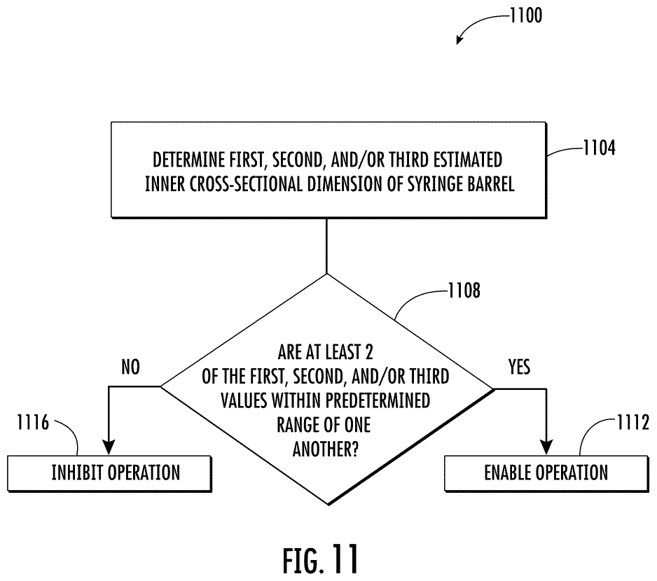

FIG. 11 illustrates an example method 1100 for confirming a size of a syringe. The method 1000 is enacted by the control unit 24 using estimated inner cross-sectional dimensions of a syringe barrel determined according to the method 700 (FIG. 7), the method 900 (FIG. 9), and/or the method 1000 (FIG. 10).

At block 1104, the method 1100 includes determining two or more estimated inner cross-sectional dimensions of the syringe barrel. The two or more estimated inner cross-sectional dimensions of the syringe barrel may be determined according to the method 700 (FIG. 7), the method 900 (FIG. 9), and/or the method 1000 (FIG. 10).

At block 1108, the method 1100 includes comparing the two or estimated inner cross-sectional dimensions of the syringe barrel to each other. For example, referring to FIG. 11, in one example of the syringe size confirmation operation, the processor 24A compares at least two of three estimated inner cross-sectional dimensions of the syringe barrel 18 to one another to determine whether the two values are within an acceptable, predetermined range of one another (e.g., within a 2-5% margin of error).

At block 1112, the method 1100 includes enabling operation of system in response to determining that two or more of the estimated inner cross-sectional dimensions of the syringe barrel are within a predetermined range of each other. For example, If a predetermined number of the values are within the acceptable range, then the loaded syringe 14 is confirmed by the processor, and the system can continue with a feeding operation. For example, as shown in FIG. 11, where the control unit 24 generates three estimated inner cross-sectional dimensions of the syringe barrel 18, such as by using the OD-based estimation operation and the two volume-based estimation operations, if two of the three values are within an acceptable, predetermined range of one another, then the processor uses those values for operating the system. For example, the processor 24 may calculate an average inner cross-sectional dimension of the acceptable values.

At block 1116, the method 1100 includes inhibiting operation of the system in response to determining that two or more of the estimated inner cross-sectional dimensions of the syringe barrel are not within a predetermined range of each other. For example, if the predetermined number of the values (e.g., two) are not within an acceptable range, then the control unit 24 may generate an error and/or will not allow the feeding operation to proceed. The system 1 may communicate to the user additional instructions, such as confirming: the syringe is properly loaded, there are no kinks in the tubing, the identified syringe is actually loaded on the holder, etc.

In one example, the automated syringe size confirmation operation comprises comparing the selected syringe model based on the OD-based syringe size estimation to one or more volume-based syringe size estimations. In this example, the volume-based syringe size estimations are used as a check on the accuracy of the syringe model. If at least one of the volume-based syringe size estimations are within an acceptable range of the syringe model, then the syringe model is used.

### Syringe Calibration Operation

In one or more embodiments, at least two estimated cross-sectional dimensions (e.g., the volume-based inner cross-sectional dimension estimation and the OD-based stored inner cross-sectional dimension of the selected model syringe) may be used for calibrating the system 1 during a syringe calibration operation.

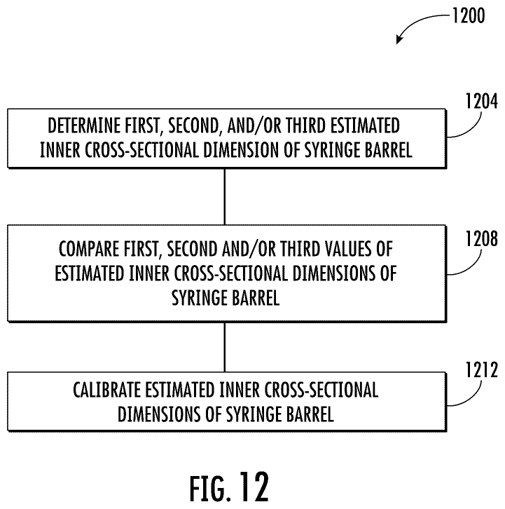

FIG. 12 illustrates an example method 1200 for calibrating the system 1 during a syringe calibration operation. The method 1200 is enacted by the control unit 24 using estimated inner cross-sectional dimensions of a syringe barrel determined according to the method 700 (FIG. 7), the method 900 (FIG. 9), and/or the method 1000 (FIG. 10).

At block 1204, the method 1200 includes determining two or more estimated inner cross-sectional dimensions of the syringe barrel. The two or more estimated inner cross-sectional diameters of the syringe barrel may be determined according to the method 700 (FIG. 7), the method 900 (FIG. 9), and/or the method 1000 (FIG. 10).

At block 1208, the method 1200 includes comparing the two or more estimated inner cross-sectional dimensions of the syringe barrel to each other.

At block 1212, the method 1200 includes calibrating the estimated cross-sectional dimensions of the syringe barrel. In one example, scaling coefficients (e.g.,

$$r = X \sqrt{\frac{V}{h π}} ,$$

where X is the scaling coefficient) can be used to statistically and accurately shift the calculated inner cross-sectional dimension (r) into known good ranges based off statistically derived test data. For example, two of the closest calculated cross-sectional inner dimensions can be used to calculate the scaling coefficient.

In another example, the control unit 24 uses two or more estimated inner cross-sectional dimensions determined using different methods to optimize the volumetric accuracy equation. For example, at block 1212, one possible method to dynamically adjust the volume accuracy equation is a three component equation contributing to the calculation of the inner cross-sectional dimension of the syringe barrel 18:

$$R = a x + b y + c z$$

Where, R is the internal syringe barrel radius; a is an inner cross-sectional dimension of a barrel of a syringe that is selected by a user and stored in memory; x is the percent contribution or weight for the constant a to contribute to the R calculation; b is the calculated R value of the model syringe based upon the outer diameter measurement; y is the percent contribution or weight for the constant b to contribute to the R calculation; c is the calculated R value based on the volume-based calculation; and z is the percent contribution or weight for the constant c to contribute to the R calculation.

The syringe calibration operation 1200 may be used during operation of the system 1 to recalibrate fluid delivery parameters due to, for example, the administration set being used. In particular, the tubing 16 of the administration set may become stretched or have wear during a period of time, whereby the accuracy of the flow rate and/or delivered volume may be affected. The volume-based estimation values may be used to recalibrate the system to ensure a more accurate delivery of fluid to the patient.

The use of multiple estimations of inner cross-sectional dimension of a loaded syringe avoids costly information and algorithmic updates along with performance inaccuracy that could occur. For example, the volume-based estimations mitigate potential errors and incurred cost of system updates within a library for constant values used for system performance whenever a syringe manufacturer changes any design parameter of their syringe such as wall thickness, outside diameter, etc. resulting in needed software updates to ensure system volume accuracy or other system algorithmic product requirements are still being met.

Additionally, advantages afforded by this detection method include real time adaptability to syringe variations and review of the individual syringe as they are loaded allowing for all syringes that can be loaded into the system to be capable of use with the system. These could come from use of different syringe sizes within a feeding process, tolerances, changes in manufacturer, manufacturing methods, material reactions or swelling, brands not currently qualified for use with the system, and various other factors.

Set forth below are various aspects of the disclosure.

Thus, one of more aspects of the present disclosure may be implemented according to one or more of the following clauses.

Clause 1. An automated fluid delivery system for delivering medical fluid to a patient, the automated fluid delivery system comprising: a flow control device including a pump and a control unit in communication with the pump; a fluid administration set including a syringe containing the medical fluid, and tubing in fluid communication with the syringe, wherein the syringe includes a barrel and a plunger, the fluid administration set being operatively coupled to the pump so that the pump generates flow of the medical fluid from the syringe through the tubing; a fluid sensor in communication with the control unit, wherein the fluid sensor is configured to detect the medical fluid in the tubing at a location downstream of the syringe; a plunger displacement sensor in communication with the control unit, wherein the plunger displacement sensor is configured to detect linear displacement of the plunger relative to the barrel as the pump generates flow of the medical fluid from the syringe through the tubing, wherein the control unit includes a processor and memory storing processor-executable instructions that, when executed by the processor, cause the processor to: determine an estimated inner cross-sectional dimension of the barrel of the syringe based on the linear displacement of the plunger and the fluid sensor detecting the medical fluid in the tubing at the location downstream of the syringe, and operate the pump based at least in part on the estimated inner cross-sectional dimension of the barrel of the syringe.

Clause 2. The automated fluid delivery system set forth in clause 1, wherein the processor-executable instructions, when executed by the processor, further cause the processor to: compare the estimated inner cross-sectional dimension of the barrel of the syringe to a second estimated inner cross-sectional dimension of the barrel of the syringe.

Clause 3. The automated fluid delivery system set forth in clause 2, wherein the second estimated inner cross-sectional dimension is based at least in part on a detected outer diameter of the syringe.

Clause 4. The automated fluid delivery system set forth in clause 2, wherein the processor-executable instructions, when executed by the processor, further cause the processor to: determine a calibrated estimated inner cross-sectional dimension of the barrel of the syringe based on the estimated inner cross-sectional dimension and the second estimated inner cross-sectional dimension; and operate the pump based at least in part on the calibrated estimated inner cross-sectional dimension of the barrel of the syringe.

Clause 5. The automated fluid delivery system set forth in clause 2, wherein the processor-executable instructions, when executed by the processor, further cause the processor to: determine if the estimated inner cross-sectional dimension of the barrel and the second estimated inner cross-sectional dimension of the barrel are within a predetermined range of one another.

Clause 6. The automated fluid delivery system set forth in clause 5, wherein the processor-executable instructions, when executed by the processor, further cause the processor to: enable operation of the pump if the estimated inner cross-sectional dimension of the barrel and the second estimated inner cross-sectional dimension of the barrel are within the predetermined range with respect to one another; and inhibit operation of the pump if the estimated inner cross-sectional dimension of the barrel and the second estimated inner cross-sectional dimension of the barrel are not within the predetermined range with respect to one another.

Clause 7. A flow control device for an automated fluid delivery system configured to deliver medical fluid to a patient, the flow control device comprising: a pump configured to be operatively coupled to a fluid administration set including a syringe containing the medical fluid, and tubing in fluid communication with the syringe, a fluid sensor in communication with the control unit, wherein the fluid sensor is configured to detect the medical fluid in the tubing at a location downstream of the syringe; a plunger displacement sensor in communication with the control unit, wherein the plunger displacement sensor is configured to detect linear displacement of the plunger relative to the barrel as the pump generates flow of the medical fluid from the syringe through the tubing, a control unit in communication with the pump, the fluid sensor, and the plunger displacement sensor, the control unit including a processor and memory storing processor-executable instructions that, when executed by the processor, cause the processor to: determine an estimated inner cross-sectional dimension of the barrel of the syringe based on the linear displacement of the plunger when the fluid sensor detects the medical fluid in the tubing at the location downstream of the syringe, and operate the pump based at least in part on the estimated inner cross-sectional dimension of the barrel of the syringe.

Clause 8. The automated fluid delivery system set forth in clause 7, wherein the processor-executable instructions, when executed by the processor, further cause the processor to: compare the estimated inner cross-sectional dimension of the barrel of the syringe to a second estimated inner cross-sectional dimension of the barrel of the syringe.

Clause 9. The automated fluid delivery system set forth in clause 8, wherein the second estimated inner cross-sectional dimension is based at least in part on a detected outer diameter of the syringe.

Clause 10. The automated fluid delivery system set forth in clause 8, wherein the processor-executable instructions, when executed by the processor, further cause the processor to: determine a calibrated estimated inner cross-sectional dimension of the barrel of the syringe based on the estimated inner cross-sectional dimension and the second estimated inner cross-sectional dimension; and operate the pump based at least in part on the calibrated estimated inner cross-sectional dimension of the barrel of the syringe.

Clause 11. The automated fluid delivery system set forth in clause 8, wherein the processor-executable instructions, when executed by the processor, further cause the processor to: determine if the estimated inner cross-sectional dimension of the barrel and the second estimated inner cross-sectional dimension of the barrel are within a predetermined range with respect to one another.

Clause 12. The automated fluid delivery system set forth in clause 11, wherein the processor-executable instructions, when executed by the processor, further cause the processor to: enable operation of the pump if the estimated inner cross-sectional dimension of the barrel and the second estimated inner cross-sectional dimension of the barrel are within the predetermined range with respect to one another; and inhibit operation of the pump if the calculated estimated inner cross-sectional dimension of the barrel and the second estimated inner cross-sectional dimension of the barrel are not within the predetermined range with respect to one another.

Clause 13. A method of estimating inner cross-sectional dimension of a barrel of a syringe for a fluid delivery system, the method comprising: pumping, via a pump, a known volume of medical fluid from a syringe through tubing connected to the syringe; detecting, via a plunger displacement sensor, linear displacement of a plunger of the syringe relative to a barrel of the syringe that corresponds to the known volume pumped from the syringe via the pump; determining an estimated inner cross-sectional dimension of the barrel of the syringe based on the linear displacement of the plunger and the known volume of medical fluid pumped from the syringe; and operate the pump based at least in part on the estimated inner cross-sectional dimension of the barrel of the syringe.

Clause 14. The method set forth in clause 13, wherein said pumping a known volume of medical fluid comprises sensing the medical fluid at a selected location of the tubing.

Clause 15. The method set forth in clause 13, further comprising comparing the estimated inner cross-sectional dimension of the barrel of the syringe to a second estimated inner cross-sectional dimension of the barrel of the syringe.

Clause 16. The method set forth in clause 15, wherein the second estimated inner cross-sectional dimension is based at least in part on a detected outer diameter of the syringe.

Clause 17. The method set forth in clause 15, further comprising: determining a calibrated estimated inner cross-sectional dimension of the barrel of the syringe based on the estimated inner cross-sectional dimension and the second estimated inner cross-sectional dimension; and operating the pump based at least in part on the calibrated estimated inner cross-sectional dimension of the barrel of the syringe.

Clause 18. The method set forth in clause 15, further comprising: enabling operation of the pump if the estimated inner cross-sectional dimension of the barrel and the second estimated inner cross-sectional dimension of the barrel are within a predetermined range with respect to one another; and inhibiting operation of the pump if the estimated inner cross-sectional dimension of the barrel and the second estimated inner cross-sectional dimension of the barrel are not within the predetermined range with respect to one another.

Clause 19. A method of estimating inner cross-sectional dimension of a barrel of a syringe loaded in a fluid delivery system, the method comprising: estimating, using a control unit, a first estimated cross-sectional dimension of the barrel of the syringe loaded in the fluid delivery system, wherein said estimating a first estimated cross-sectional dimension of the barrel is based on a detected first parameter analyzed by the control unit; estimating, using the control unit, a second estimated cross-sectional dimension of the barrel of the syringe loaded in the fluid delivery system, wherein said estimating a second estimated cross-sectional dimension of the barrel is based on a detected second parameter, different from the first parameter, analyzed by the control unit; comparing the first and second estimated cross-sectional dimensions to one another; and performing an operation of the fluid delivery system, using the control unit, based on the comparison of the first and second estimated cross-sectional dimensions.

Clause 20. The method set forth in clause 19, wherein said estimating a first estimated cross-sectional dimension of the barrel comprises calculating an estimated inner cross-sectional dimension of the barrel of the syringe based on linear displacement of a plunger corresponding to a known volume of medical fluid pumped from the syringe.

Embodiments may be described in the general context of computer-executable or processor-executable instructions, such as program modules, executed by one or more computers or other devices. The computer-executable instructions may be organized into one or more computer-executable components or modules including, but not limited to, operations, programs, objects, components, and data structures that perform particular tasks or implement particular abstract data types. Aspects may be implemented with any number and organization of such components or modules. For example, various features or aspects are not limited to the specific computer-executable instructions or the specific components or modules illustrated in the figures and described herein. Other embodiments may include different computer-executable instructions or components having more or less functionality than illustrated and described herein.

Further, the order of execution or performance of the operations in any of the embodiments illustrated and described herein is not essential, unless otherwise specified. That is, the operations may be performed in any order, unless otherwise specified, and embodiments may include additional or fewer operations than those disclosed herein. For example, it is contemplated that executing or performing a particular operation before, contemporaneously with, or after another operation is within the scope of one or more aspects.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the disclosure are achieved and other advantageous results attained.

As various changes could be made in the above constructions without departing from the scope of the disclosure, it is intended that all matter contained in the above description and shown in the accompanying drawings shall be interpreted as illustrative and not in a limiting sense.

1. An automated fluid delivery system for delivering medical fluid to a patient, the automated fluid delivery system comprising:
a flow control device including a pump and a control unit in communication with the pump;
a fluid administration set including a syringe containing the medical fluid, and tubing in fluid communication with the syringe, wherein the syringe includes a barrel and a plunger, the fluid administration set being operatively coupled to the pump so that the pump generates flow of the medical fluid from the syringe through the tubing;
a fluid sensor in communication with the control unit, wherein the fluid sensor is configured to detect the medical fluid in the tubing at a location downstream of the syringe;
a plunger displacement sensor in communication with the control unit, wherein the plunger displacement sensor is configured to detect linear displacement of the plunger relative to the barrel as the pump generates flow of the medical fluid from the syringe through the tubing,
wherein the control unit includes a processor and memory storing processor-executable instructions that, when executed by the processor, cause the processor to:
determine an estimated inner cross-sectional dimension of the barrel of the syringe based on the linear displacement of the plunger and the fluid sensor detecting the medical fluid in the tubing at the location downstream of the syringe, and
operate the pump based at least in part on the estimated inner cross-sectional dimension of the barrel of the syringe.

2. The automated fluid delivery system set forth in claim 1, wherein the processor-executable instructions, when executed by the processor, further cause the processor to: compare the estimated inner cross-sectional dimension of the barrel of the syringe to a second estimated inner cross-sectional dimension of the barrel of the syringe.

3. The automated fluid delivery system set forth in claim 2, wherein the second estimated inner cross-sectional dimension is based at least in part on a detected outer diameter of the syringe.

4. The automated fluid delivery system set forth in claim 2, wherein the processor-executable instructions, when executed by the processor, further cause the processor to:
determine a calibrated estimated inner cross-sectional dimension of the barrel of the syringe based on the estimated inner cross-sectional dimension and the second estimated inner cross-sectional dimension; and
operate the pump based at least in part on the calibrated estimated inner cross-sectional dimension of the barrel of the syringe.

5. The automated fluid delivery system set forth in claim 2, wherein the processor-executable instructions, when executed by the processor, further cause the processor to:
determine if the estimated inner cross-sectional dimension of the barrel and the second estimated inner cross-sectional dimension of the barrel are within a predetermined range of one another.

6. The automated fluid delivery system set forth in claim 5, wherein the processor-executable instructions, when executed by the processor, further cause the processor to:
enable operation of the pump if the estimated inner cross-sectional dimension of the barrel and the second estimated inner cross-sectional dimension of the barrel are within the predetermined range with respect to one another; and
inhibit operation of the pump if the estimated inner cross-sectional dimension of the barrel and the second estimated inner cross-sectional dimension of the barrel are not within the predetermined range with respect to one another.

7. A flow control device for an automated fluid delivery system configured to deliver medical fluid to a patient, the flow control device comprising:
a pump configured to be operatively coupled to a fluid administration set including a syringe containing the medical fluid, and tubing in fluid communication with the syringe,
a fluid sensor in communication with a control unit, wherein the fluid sensor is configured to detect the medical fluid in the tubing at a location downstream of the syringe;
a plunger displacement sensor in communication with the control unit, wherein the plunger displacement sensor is configured to detect linear displacement of the plunger relative to the barrel as the pump generates flow of the medical fluid from the syringe through the tubing,
the control unit in communication with the pump, the fluid sensor, and the plunger displacement sensor, the control unit including a processor and memory storing processor-executable instructions that, when executed by the processor, cause the processor to:
determine an estimated inner cross-sectional dimension of the barrel of the syringe based on the linear displacement of the plunger when the fluid sensor detects the medical fluid in the tubing at the location downstream of the syringe, and
operate the pump based at least in part on the estimated inner cross-sectional dimension of the barrel of the syringe.

8. The automated fluid delivery system set forth in claim 7, wherein the processor-executable instructions, when executed by the processor, further cause the processor to: compare the estimated inner cross-sectional dimension of the barrel of the syringe to a second estimated inner cross-sectional dimension of the barrel of the syringe.

9. The automated fluid delivery system set forth in claim 8, wherein the second estimated inner cross-sectional dimension is based at least in part on a detected outer diameter of the syringe.

10. The automated fluid delivery system set forth in claim 8, wherein the processor-executable instructions, when executed by the processor, further cause the processor to:
determine a calibrated estimated inner cross-sectional dimension of the barrel of the syringe based on the estimated inner cross-sectional dimension and the second estimated inner cross-sectional dimension; and
operate the pump based at least in part on the calibrated estimated inner cross-sectional dimension of the barrel of the syringe.

11. The automated fluid delivery system set forth in claim 8, wherein the processor-executable instructions, when executed by the processor, further cause the processor to:
determine if the estimated inner cross-sectional dimension of the barrel and the second estimated inner cross-sectional dimension of the barrel are within a predetermined range with respect to one another.

12. The automated fluid delivery system set forth in claim 11, wherein the processor-executable instructions, when executed by the processor, further cause the processor to:
enable operation of the pump if the estimated inner cross-sectional dimension of the barrel and the second estimated inner cross-sectional dimension of the barrel are within the predetermined range with respect to one another; and
inhibit operation of the pump if the estimated inner cross-sectional dimension of the barrel and the second estimated inner cross-sectional dimension of the barrel are not within the predetermined range with respect to one another.

13. A method of estimating inner cross-sectional dimension of a barrel of a syringe for a fluid delivery system, the method comprising:
pumping, via a pump, a known volume of medical fluid from a syringe through tubing connected to the syringe;
detecting, via a plunger displacement sensor, linear displacement of a plunger of the syringe relative to a barrel of the syringe that corresponds to the known volume pumped from the syringe via the pump;
determining an estimated inner cross-sectional dimension of the barrel of the syringe based on the linear displacement of the plunger and the known volume of medical fluid pumped from the syringe; and
operate the pump based at least in part on the estimated inner cross-sectional dimension of the barrel of the syringe.

14. The method set forth in claim 13, wherein said pumping a known volume of medical fluid comprises sensing the medical fluid at a selected location of the tubing.

15. The method set forth in claim 13, further comprising comparing the estimated inner cross-sectional dimension of the barrel of the syringe to a second estimated inner cross-sectional dimension of the barrel of the syringe.

16. The method set forth in claim 15, wherein the second estimated inner cross-sectional dimension is based at least in part on a detected outer diameter of the syringe.

17. The method set forth in claim 15, further comprising:
determining a calibrated estimated inner cross-sectional dimension of the barrel of the syringe based on the estimated inner cross-sectional dimension and the second estimated inner cross-sectional dimension; and
operating the pump based at least in part on the calibrated estimated inner cross-sectional dimension of the barrel of the syringe.

18. The method set forth in claim 15, further comprising:
enabling operation of the pump if the estimated inner cross-sectional dimension of the barrel and the second estimated inner cross-sectional dimension of the barrel are within a predetermined range with respect to one another; and
inhibiting operation of the pump if the estimated inner cross-sectional dimension of the barrel and the second estimated inner cross-sectional dimension of the barrel are not within the predetermined range with respect to one another.

19. (canceled)

20. (canceled)
